(19) United States
(12) Patent Application Publication
(10) Pub. No.: US 2026/0194508 A1
(43) Pub. Date: Jul. 9, 2026

(54) System and Method for Carbon Monitoring and Management in Water Systems

(71) Applicant: Vycarb Inc., Brooklyn, NY (US)

(72) Inventor: Frank Garrett Boudinot, Brooklyn, NY (US)

(21) Appl. No.: 19/133,924

(22) PCT Filed: Dec. 22, 2023
(86) PCT No.: PCT/US2023/085767

§ 371 Date: May 29, 2025

Related U.S. Application Data
(60) Provisional application No. 63/434,760, filed on Dec. 22, 2022.

(51) Int. Cl. G01N 33/18

(52) U.S. Cl. CPC G01N 33/1846

(57) ABSTRACT

A system and method for carbon monitoring in water systems that can include: at a first monitoring system, enclosing water within a receptacle of the first monitoring system, with the water at a filled level that leaves a defined gas portion within the receptacle; collecting sensor data from the water and the defined gas portion comprising: measuring carbon concentration of the gas portion, and measuring water conditions including at least water temperature, water conductivity, and water pH; after the water reaches an equilibrium state within the receptacle, determining dissolved inorganic carbon concentration state based on the carbon concentration, temperature, water conductivity, and water pH. The system and method may additionally include adding a supplement to the water system and regulating the adding of the supplement based on the determined change in dissolved inorganic carbon concentration states.

### CROSS-REFERENCE TO RELATED APPLICATIONS

This application claims the benefit of U.S. Provisional Application No. 63/434,760, filed on 22 Dec. 2022, which is incorporated in its entirety by this reference.

### TECHNICAL FIELD

This invention relates generally to the field of carbon monitoring, management, and storage, and more specifically to a new and useful system and method integrating carbon management, storage, and monitoring in an aqueous solution.

### BACKGROUND OF THE INVENTION

With climate change becoming a bigger issue on a daily basis, the control over carbon emissions has become an important topic for research and development. Although most technologies deal with reducing carbon in the air, a significant amount of carbon emissions come from water, which has been left relatively ignored. As one problem, there is not a way of accurately quantifying and verifying carbon reduction processes within water systems. Thus, there is a need in the field of carbon management to create a new and useful system and method for carbon monitoring in water systems. This invention provides such a new and useful system and method.

### BRIEF DESCRIPTION OF DRAWINGS

FIGS. 1A and 1B are schematic representations of a top and side view of one system variation.

FIGS. 2A and 2B are schematic representations of another system variation with a detachable lid or cover for adding of water.

FIG. 3 is a schematic representation of an example automated system.

FIG. 4 is a schematic representation of a second example automated system.

FIG. 5 is a schematic representation of an example automated system with a carbon manipulation system.

FIG. 6 is a schematic representation of a variation using a supplement of CO₂.

FIG. 7 is a schematic representation of a system with multiple monitoring systems.

FIG. 8 is a schematic representation of a variation using a supplement of an alkaline source.

FIG. 9 is a schematic representation of one carbon manipulation system.

FIG. 10 is a detailed schematic representation of operational components of a variation of a carbon manipulation system.

FIG. 11 is a flowchart representation of an example method.

FIG. 12 is a flowchart representation of a method variation.

FIG. 13 is a flowchart representation of a method variation used in modifying water quality.

FIG. 14 is a flowchart representation of a method variation using convergent analysis of different methods for determining dissolved inorganic carbon compounds.

FIG. 15 is a schematic representation of method applied within a multi-monitoring system implementation.

FIG. 16 is a detailed flow diagram of sensing data and determination of dissolved inorganic carbon concentration state.

FIG. 17 is a chart depicting application of convergent analysis of multiple methods for modeling dissolved inorganic carbon compounds.

FIG. 18 is exemplary sensor data and dissolved inorganic carbon concentration state data resulting from modification of water conditions.

FIG. 19 is an exemplary system architecture that may be used in implementing the system and/or method.

### DETAILED DESCRIPTION OF THE EMBODIMENTS

The following description of the embodiments of the invention is not intended to limit the invention to these embodiments but rather to enable a person skilled in the art to make and use this invention.

### 1. Overview

A system and method for carbon monitoring in a water system leverages a monitoring system using a monitoring approach of an enclosed water sample that reaches equilibrium to measure and model carbon-based compound concentrations in an aqueous/water system. The system and method can use a detection receptacle, that includes a spectrometric sensor enabled to measure gaseous carbon compounds and sensors to measure the water system properties, comprising: partially filling the detection receptacle with water; equilibrating the unfilled region and the filled region of the detection receptacle; using the spectrometric sensor, measuring the carbon concentration of the unfilled region, measuring the water properties includes temperature, pH (and other pH properties such as acidity and alkalinity), electrical conductivity, and salinity of the water; and determining the dissolved inorganic carbon (DIC) concentrations using carbon concentrations of the unfilled region and water chemistry of the filled region to determine carbon concentrations of filled region based on carbon equilibrium relationship between air and water. The system and method may be used in outputting or using DIC concentration state information, alternatively characterized as water carbon concentration. DIC state information can include concentration measurements or other quantifications of one or more different DIC compounds (or DIC species) such as CO₂, bicarbonate (HCO₃), and carbonate (CO₃) compounds/ions in a liquid/water, as well as associated carbon chemistry parameters such as carbonate saturation state.

In some variations, the system and method may be used in combination with a carbon manipulation system that can be used to augment or modify water systems to initiate the removal of CO₂ through conversion into stable forms within a water system.

In one particular variation, the system and method may be used to enable a measurement-controlled alkalinity addition system for carbon dioxide (CO₂) removal and storage in water (“CDR System”) that converts excess biogenic, dissolved CO₂ in high CO₂ waters (e.g., estuaries) bound for the atmosphere into stable, dissolved bicarbonate (HCO₃) and carbonate (CO₃) ions in the same water. In this way, the system and method may reduce excess CO₂ in waters, which provides an important ecosystem co-benefit by reducing harmful local acidification. The excess CO₂ (e.g., >425 ppm) is detected using the monitoring systems, then a carbon manipulation system/CDR system may add amounts of alkaline solution until reduction of CO₂ reaches ambient concentrations, (~425 ppm) with associated pH and saturation state endpoint controls.

The system and method may be generally implemented as part of any closed or open water source, for water monitoring and management. For closed systems, the system and method may be used to monitor and maintain water quality (e.g., reservoirs). For open systems, the system and method may enable fluid management in conjunction with other activities that occur on that body of water. For example, the system and method may be implemented in regions where waste is released, areas with variations in human population use of the water (e.g., tourist areas), near mining operations, and other factory operations and waste disposal.

In one example, the system and method may be used in connection with a water way such as a river or stream. The system and method may be used for monitoring and active adjustment of water conditions within the water way.

In another example, the system and method may be used within an industrial water system, such as a mining operation. In this variation, the system and method may be used for treating the water used in the mining operation and in some variations, also used for storing carbon.

The system and method may provide a number of potential benefits. The system and method are not limited to always providing such benefits and are presented only as exemplary representations for how the system and method may be put to use. The list of benefits is not intended to be exhaustive and other benefits may additionally or alternatively exist.

As one potential benefit, the system and method can provide measurement-based verification for carbon capture within water systems. How alkalinity behaves in an ecosystem can differ drastically from theory and in-lab experimentation. There can be various carbon removal and/or storage approaches in water, including the addition of alkalinity to either increase absorption of CO₂ by water or convert dissolved CO₂ to HCO₃ and CO₃. The system and method described herein can provide a way of measuring efficacy of carbon dioxide removal efforts and/or to manage operation of such carbon removal and storage processes.

As another potential benefit, the system and method may be implemented as a relatively low cost and robust way of monitoring DIC concentrations. Once CO₂ enters water, DIC speciation in water can occur making it challenging to quantify and measure. Traditional approaches rely on laboratory analysis, which is time consuming and expensive, and/or rely on expensive sensing systems that make use of expensive components like gas-permeable membranes and internal calibration gasses. Such approaches have prohibitively high costs. This can make enable monitoring system that can practically be distributed and used in practice.

As another potential benefit, the system and method can operate substantially autonomously, which can help to keep operational costs low and make it a practical solution.

The system and method may provide the benefit of more precise carbon information. Through use of spectrometric sensors, the system and method may be enabled to provide measurements of carbon dioxide, carbonate, and bicarbonate.

Additionally, the system and method may provide the benefit of real time continuous carbon measurements. Through the implementation of the automated variations of the system, continuous measurements of carbon concentrations may be collected and directly used.

As another potential benefit, the system and method may provide a solution for actively managing carbon capture by actively modifying the alkalinity and/or other properties of a water system. In some variations, carbon could be stored within the water and/or the carbon present in the water may be altered for enhanced storage. Such changes could be controlled using the active monitoring of the system and method.

Additionally, the system and method may further enable monitoring and managing carbon storage. In conjunction with monitoring and managing water quality, this would provide the potential benefit of enabling improved water quality (e.g., by reducing water acidity).

### 2. System

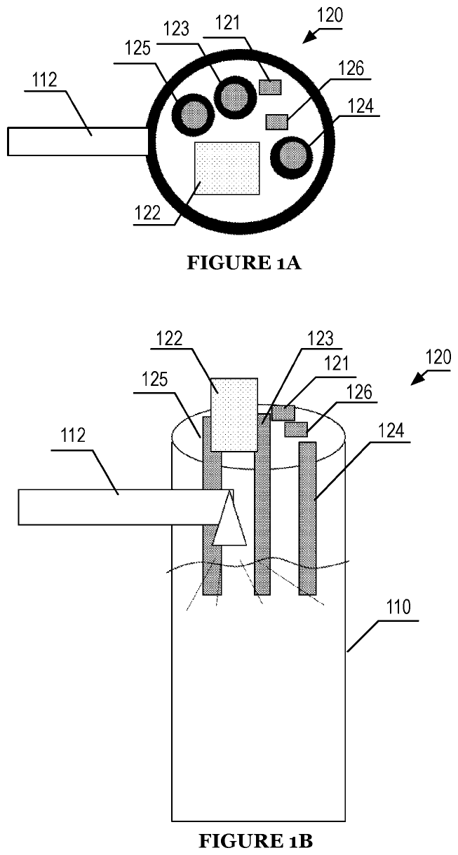

As shown in FIGS. 1A and 1B, 2A and 2B, 3, and 4, a system for carbon monitoring of a fluid may include: a measuring receptacle 110, wherein once partially filled with a liquid, in an open state, allows the liquid to flow into and out of the measuring receptacle, and in a closed or operating state, prevents gaseous flow into or out of the measuring receptacle; and a sensor system 120 integrated to detect conditions of the water/aqueous portion and a gas portion. The sensor system preferably includes a pressure sensor 121 and a spectroscopic sensor 122 for monitoring the gas portion and a temperature sensor 123, a conductivity sensor 124, and a pH sensor 125 for monitoring the water portion. Such a system may be implemented within a single monitoring system. In some alternative variations, multiple instances of such monitoring systems may be used in combination such that DIC concentration state data can be collected at multiple points and/or across regions.

The system when implemented as a single monitoring instance 100 functions as a monitoring device, that once partially filled with the liquid and in a closed state, detects and measures the carbon content of the liquid. That is, the system leverages general properties of the liquid (e.g., salinity, pH, and temperature) in conjunction with gaseous carbon concentration measurements in the non-filled region of measuring receptacle (i.e., the gas portion), to determine the liquid carbon concentration. Thus, in many variations, the sensor system 120 may further include other sensor components enabled to further measure gas and/or liquid properties within the measuring receptacle.

The system may be particularly useful for measurement for or monitoring of carbon compound concentrations in water. As a major contributor of carbon in water is carbon dioxide, the system enables measuring and monitoring of carbon dioxide in water. In the same manner, the system may be implemented to measure carbonate (CO₃) and bicarbonate (HCO₃) concentrations (or their conjugate acids, carbonic acid (H₂CO₃)). Generally, the system may be implemented to measure fluctuations of these compounds (or other carbon compounds) in any fluid, and potentially determine the specific concentrations of each of these carbon compounds within the fluid.

In some variations, the monitoring system may operate fully or partially autonomously, wherein the monitoring system can be setup for monitoring in a specific region and then can continuously or periodically monitor DIC concentrations at that site.

In some alternative variations, the monitoring system may determine DIC concentration state of the water (e.g., measuring dissolved carbon amounts for CO₂, CO₃, and/or HCO₃) in response to a measurement request. For example, a user interface may trigger a measurement request, and the system can collect measurements and output the determined DIC concentration state data.

In some variations like the automated variations, the system may further include: a communication system 130, that enables system communication with external systems and users and includes the ability to transmit collected data to an external source; and a control system 140, that monitors the system components and changes system operation parameters as desired by implementation and operational use.

In some examples, the automated system variation may further be part of a larger control feedback system. For example, the automated system may work in conjunction with a carbon manipulation system 150 (that may or may not be a part of the system). Accordingly, some variations the system may include or interface with a carbon manipulation system 150. The carbon manipulation system 150 may function to increase, or decrease, carbon concentration in the fluid through modification of the water. In some variations for increasing carbon concentration, such as in a mining use case example, CO₂ could be added to the water. In conjunction, the automated system may monitor changes of the carbon concentration of the fluid. For some pre-determined desired carbon concentration range, the automated system may provide a control feedback to the carbon manipulation system 150; whereas by leveraging the collected carbon concentration data, the automated system may direct the carbon manipulation system 150 to increase or decrease the fluid carbon concentration. In many implementations, the automated system may further provide the carbon manipulation system 150 a control feedback to modify the rate of increase or the rate of decrease of carbon.

The control feedback system may be an open or closed system. As part of a closed system, the carbon manipulation system 150 may be situated on, or within, the measuring receptacle 110. The measuring receptacle 110 may be filled with some desired volume of fluid (e.g., water). The carbon therein may then be modified until the desired carbon content is reached based on the measurement of the fluid in the receptacle. Once the desired concentration is reached, the fluid may be released for new fluid to enter and be modified, or may be stored within the receptacle.

Closed system variations may use a specifically designed measuring receptacles 110 (e.g., with a desired fluid capacity), or may be incorporated as part of previously existing fluid storage systems. For example, water silos and other closed water reservoirs (e.g., at water processing plants) may be modified into a closed control feedback system.

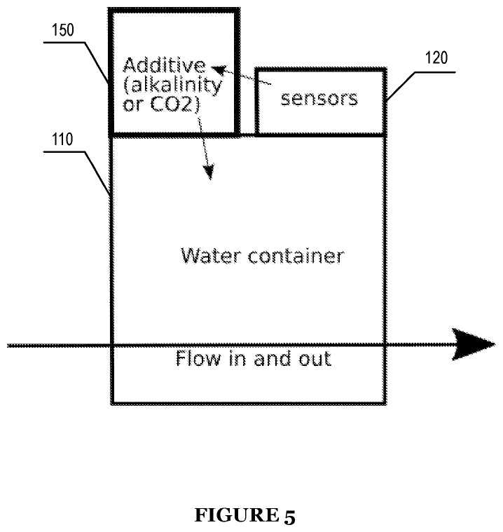

In an automated example of a closed system, as shown in FIG. 5, the carbon manipulation system 150 modifies the carbon concentration within the measuring receptacle 110 in direct response to sensor measurements of the fluid in the receptacle. In one implementation of this example, the carbon manipulation system 150 may contain stored carbon (e.g. carbon dioxide). In response to high pH, high alkalinity measurements in the fluid, the carbon manipulation system 150 may release the stored carbon dioxide. Alternatively, in response to high CO₂, low pH measurements of the fluid, the carbon manipulation system 150 may introduce CO₂ removing minerals or materials (e.g., alkaline hydroxides like magnesium hydroxide (Mg(OH)2,), carbonates like limestone (CaCO₃), alkaline silicates like wollastonite (CaSiO3), and sorbents like zeolites). As the general trends for a certain region are typically constant, the carbon manipulation system 150 may typically store either carbon reducing/pH increasing compounds or carbon depositing/pH decreasing compounds. But this is only a desired specific limitation to limit costs and space. Generally, the carbon manipulation system 150 may actively store both carbon increasing and decreasing compounds, which may be implemented for use cases with larger fluctuations in water conditions.

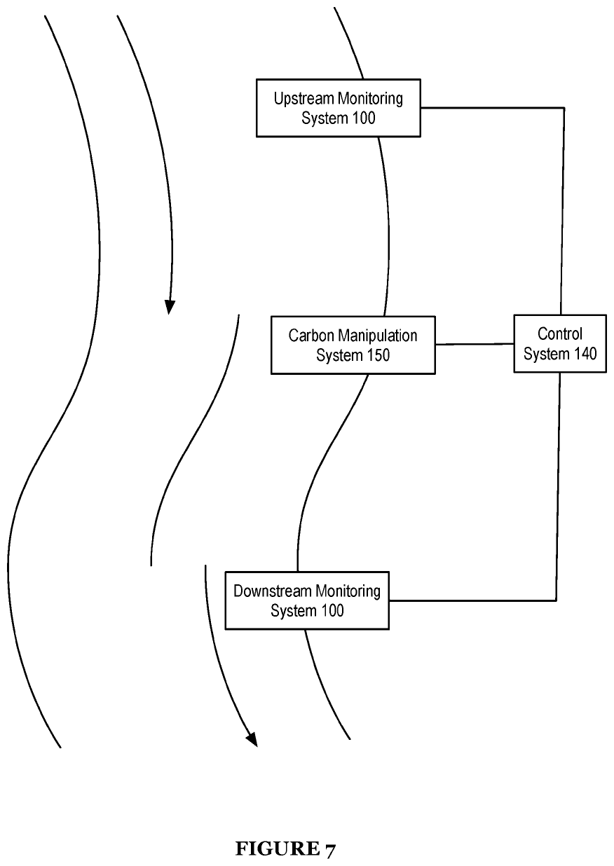

In one implementation of the control feedback example. A carbon manipulation system 150 may be setup along a river (or other body of moving water) to decrease the carbon dioxide concentration of the river as shown in FIG. 7. An automated monitoring system may be setup downstream (e.g., 100 meters) from the carbon manipulation system 150. Generally, the automated monitoring system may be set anywhere downstream, where the precise location may be dependent on the desired implementation. For example, in one implementation the automated monitoring system may be set adjacent to the carbon manipulation system 150 and in another implementation the automated monitoring system may be situated several miles downstream of the carbon manipulation system 150 (e.g., along a narrow water byway). As the carbon dioxide of the water is decreased, the automated monitoring system may provide control feedback to the carbon manipulation system 150 to prevent the water carbon dioxide levels to reach a level that would harm local water flora. The automated monitoring system may also provide feedback in response to concentrations of other forms of carbon (e.g., HCO₃, CO₃, etc.). In these variations, automated monitoring system may release CO₂ into the system, thereby remediating the alkalinity or acidity of the water.

In another implementation of the control feedback example, the system may include at least two automated monitoring systems. In a similar flow implementation as described above, one automated monitoring system may be situated upstream of the carbon manipulation system 150 and another automated monitoring system may be situated downstream from a carbon manipulation system 150. In this implementation, the upstream unit may function as a control, or as a first measuring unit. Measurements from the upstream unit may be used (e.g., by the control system) to determine the quantity of carbon that should be reduced using the carbon manipulation system 150. The monitoring unit downstream, may then function as a final, or second measuring unit. The second measuring unit may then assess the effectiveness of carbon manipulation and (e.g., through the control system) provided a finer modification to carbon concentration by the carbon manipulation system 150.

In a third implementation of the control feedback example, the system may comprise a network of carbon monitoring systems. In this example each carbon monitoring system may be automated. Alternatively, any number of systems in the network may be manually implemented. This implementation may be particularly useful in regions where water flow is inconsistent, or unknown. In the network example, a network of carbon monitoring systems may be situated anywhere in an interconnected water pathway with the carbon manipulation system 150 (e.g., bays, lakes, channels, etc.). As the carbon manipulation system 150 modifies the DIC concentration, measurement at each carbon monitoring system (or node) may be used to monitory and modify operation of the carbon manipulation system 150. Dependent on operation conditions (e.g., flow, temperature, water biome, etc.), this may lead to many types of varied operation. In one example, each monitoring system may control that a local region carbon concentration does not rise above, or fall below, a certain threshold. In another example, the carbon manipulation system 150 is provided with operational instructions that is the result of the average of the entire network. As the significance of each node becomes clearer, this average may be weighted by the node significance. In another example, nodes most proximal to the carbon manipulation unit may serve as safety controls (e.g., prevent extreme carbon conditions), while nodes more distant may provide measurement of operational efficacy (e.g., provided finer control feedback).

The measuring receptacle 110 functions as the container where carbon concentrations are measured. The measuring receptacle 110 comprises a container which may be filled with water to be functionally divided into two portions: a top portion (also referred to as the gas portion); and a bottom portion (also referred to as the liquid or water portion). As used herein, the terms top portion and bottom portion (or gas and liquid portions) are generally functional designations that suggest a level that the measuring receptacle should be partially filled to function. That is, the top and bottom portion do not necessarily suggest a different construction between the top and bottom portion, but an approximate level that the measuring receptacle needs to be filled with a liquid to function.

The measuring receptacle 110 preferably includes an internally defined cavity, that when measuring water can be filled partially with water. The receptacle preferably has a shape profile such that when filled with water to target level, the gas portion is a continuously connected area.

In some variations, particularly closed control feedback implementations, the measuring receptacle 110 may comprise a previously existing fluid storage container that is modified to serve as a system component. Generally, the measuring receptacle 110 may comprise any fluid containing container. Examples include: silos and closed reservoirs.

The measuring receptacle 110 may be composed of any material that is (or can be made) water and liquid impermeable (e.g., plastics or metals). Dependent on implementation (e.g., fluid to be examined), the measuring receptacle 110 may be made of material that is corrosion resistant to the fluid.

The measuring receptacle 110 may have at least one opening. Dependent on implementation, the number and type of openings may be significantly different.

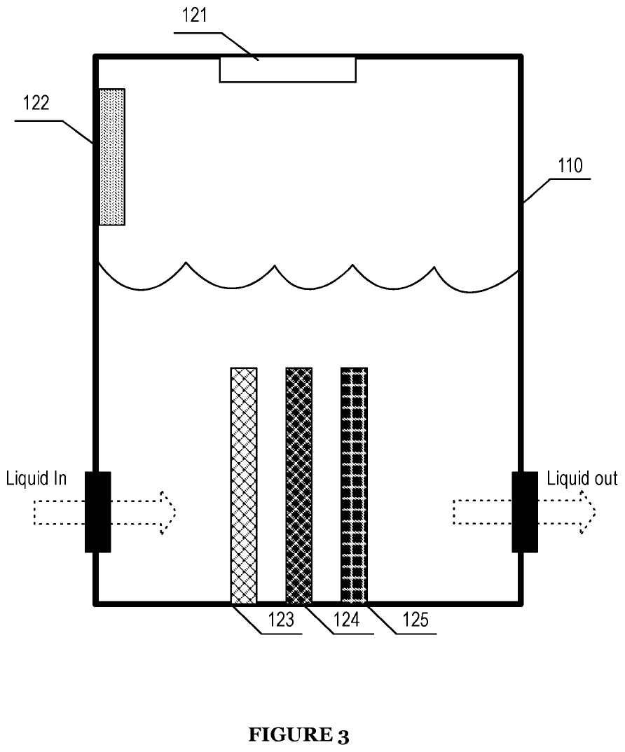

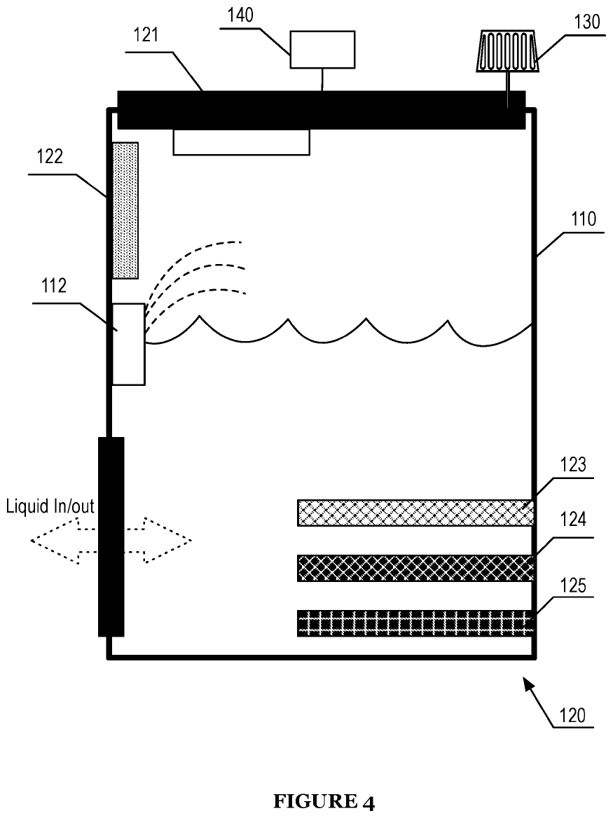

The measuring receptacle 110 preferably includes the sensor system 120 integrated into it with sensors of the sensor system 120 configured to read appropriate attributes of the environment within the receptacle 110. As shown in FIGS. 1A and 1B, the sensors may be integrated into a top portion configured to be adjacent to the gas portion when filled (e.g., in the headspace region of the device). Sensors for monitoring the water can extend or otherwise be positioned to contact the water. Sensors for monitoring the gas portion can be oriented to be within the gas portion. As shown in FIGS. 3 and 4, other arrangements and configuration of the different sensors of the sensor system 120 may alternatively be used within the measuring receptacle 110.

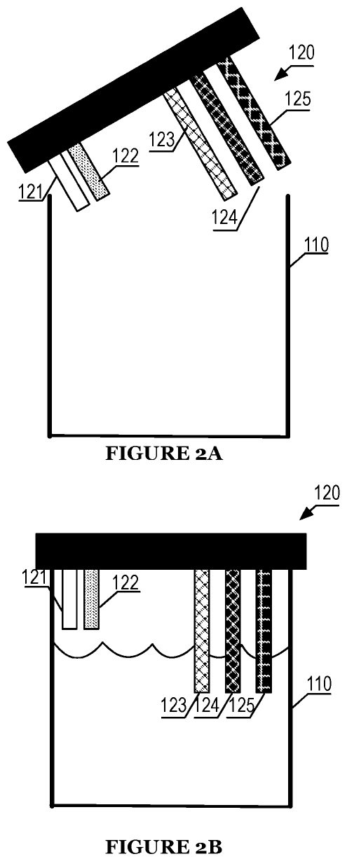

The measuring receptacle 110 may include a cap with integrated sensors from 120. In one example, as shown in FIGS. 2A and 2B, the opening may comprise a detachable lid, such that the lid may be detached, the measuring receptacle 110, partially filled with water, and then the lid reattached for operation. In other variations, the measuring receptacle 110 may include automated doors that can be automatically opened and closed. The doors preferably include seals such that when in a closed state the contained water can be enclosed within the measuring receptacle 110.

The measuring receptacle 110 or the monitoring system more generally may include a controllable air valve that can be opened to allow air flow between the gas portion and the outside air and closed to stop air flow. The air valve may be opened to facilitate emptying of water or removing water from the receptacle. The air valve may be closed when the receptacle is refilled to seal or isolate the gas portion when establishing an equilibrium.

In another example, as shown in FIG. 3, for an automated operation, the measuring receptacle 110 may have an inlet and outlet such that liquid and flow through the receptacle. Dependent on implementation, these openings may be permanently open, or closable. A pump or other system may be included to control the intake and output of liquid from the receptacle 110. This may be particularly useful for an implementation in a high flow area.

In another automated example, as shown in FIG. 4, the measuring receptacle may have a single opening for liquid exchange. Additionally, the measuring receptacle may have an additional “lid-like” opening. This lid-like opening may enable easy accessibility to the internal components of the measuring receptacle for easier cleaning and servicing.

Generally, the measuring receptacle 110 may have any number of openings, both in the liquid or gas portion of the receptacle. Depending on implementation, these openings may be closable or permanently open. In some variations, during a closed and operating state, the door or opening for water flow into and/or out of the measuring receptacle 110 may be sealed such that there is no gas exchange between the gas portion of the receptacle and the exterior of the receptacle. Note, this does not require for any opening to actually be closed. For example, as shown in FIG. 3, the two openings may be situated in the liquid portion of the measuring receptacle 110, such that the liquid itself sufficiently prevents gas exchange with the exterior.

In some variations, the system may additionally include a sprayer, mixer, or other perturbation system 112 to perturb the water. Disturbance or mixing of water may be performed upon entrance (e.g., while filling the receptacle) or after the water has reached a filled level. This may accelerate establishing an equilibrium.

The sensor system 120 functions to collect sensor data for the gas and liquid portion.

The sensor system may include a pressure sensor 121 and a spectroscopic sensor 122 for monitoring the gas portion and a temperature sensor 123, a conductivity sensor 124, and a pH sensor 125 for monitoring the water portion.

The sensors of the sensor system 120 are preferably situated to measure properties of the appropriate portion of the measuring receptacle 110 (i.e., gas sensors are situated in the gas portion and liquid sensors are situated in the liquid portion). Examples of gas sensors that may be included in the sensor system 120 include: temperature sensor, pressure sensor, hygrometer. Examples of liquid sensors that may be included in the sensor system 120 include: temperature sensor, pressure sensor, pH sensor, alkalinity sensor, flow meter, electrical conductivity sensor, refraction sensor (e.g., refractometer), specific gravity sensor (e.g., hydrometer), etc. Dependent on the liquid for measurement and other environmental conditions any other sensor may be incorporated as part of the sensor system. Sensors of the sensor system 120 may function to measure the specific quantities they are designed for but may also work in conjunction to determine more complex fluid properties, such as viscosity and salinity.

The pressure sensor 121 functions to measure the pressure in the gas portion. This can be used to control the partial pressure of the unfilled portion through connection with an electrically controlled air valve, ensuring maintained gas pressure of the unfilled region during and after active filling of water in the container. This can also provide refined quantitation of the gas pressure which is needed to calculate water carbon using carbon sensing of the air.

The spectroscopic sensor 122 functions to determine the concentration of CO₂ or other carbon compounds in the gas portion. The spectroscopic sensor 122 is preferably attuned and positioned to measure carbon compounds in the gas portion of the measuring receptacle 120. More specifically, the spectroscopic sensor 122 is attuned to detect and measure common carbon compounds in gas, such as carbon dioxide. In one example, the spectroscopic sensor 122 is a non-dispersive infrared (NDIR) sensor. The sensor can also measure temperature and humidity for self-calibration, or the sensor can be calibrated using external humidity and temperature readings from the rest of the sensor platform. Other sensor types may alternatively be used.

In some variations, the system may additionally include a fan integrated into the measuring receptacle 110 to circulate the air. This may function to improve measurement quality and possible accelerate achievement of equilibrium.

The temperature sensor 123 functions to measure the temperature of the water. In some variations, the temperatures sensor 123 can be a separate sensor that measures the temperature of the water for improved accuracy of the final calculated water carbon conditions. In some variations, the temperature sensor 123 may be part of the CO₂ sensor like the Spectroscopic sensor 122.

The conductivity sensor 124 functions to measure electrical conductivity of the water. The conductivity may be used to determine a measurement of the alkalinity and/or the salinity of the water.

The pH sensor 125 functions to measure the pH level of the water.

Other additional or alternative sensors may also be used. In one variation, a depth sensor may be used to determine the level of the water.

In some variations, particularly automated variations, the system may include a communication system 130. The communication system 130 functions to enable system communication of the system with any external device(s). The communication system 130 may be situated anywhere on, within or in connection with the system. In some variations, the communication system 130 may have components (e.g., an antenna) situated on the exterior and/or gas portion of the measuring receptacle to better enable signal transfer.

In some implementations, the communication system 130 may include only simple one-directional data transfer (e.g., the system uni-directionally transmits gather data to an external device). Alternatively, the communication system 130 may enable signals and/or data may to be sent between the system and any external devices. Examples include: smart phone, computer, remote server, etc. Depending on the range, type of data transfer, and type of external device(s), the communication system 130 may incorporate any type of wireless technology and protocol. Examples include: Wi-Fi® or Bluetooth®, RF, IR, and any wireless cellular technology (2G, 3G, 4G, etc.).

The system may include a control system 140. The control system 140 may be particularly suited for automated variations of the system but may be included in any desired implementation. The control system 140 may function to change operating parameters of the system. Changes of the operating parameters of the system may be in response to data feedback (e.g., the controlled feedback during carbon manipulation as mentioned above, weather conditions), or in response to external input received (e.g., user input through the communication system).

The control system may include one or more computer-readable mediums storing instructions that, when executed by the one or more computer processors, cause a computing platform to perform operations comprising: enclosing water within a receptacle of the first monitoring system, with the water at a filled level that leaves a defined gas portion within the receptacle; collecting sensor data from the water and the defined gas portion; after the water reaches an equilibrium state within the receptacle, determining DIC concentration state based on the sensor data of the water and defined gas portion. The instructions may additionally or alternatively be configured to perform any of the variations of the method processes described herein.

Dependent on implementation, the system may have many operating parameters that may be modified through the control system 140. For example, the control system 140 may enable activation (or deactivation) of the system and activate certain operating modes. Possible operating parameters of the system may include: turning on the system, turning off the system, modifying the liquid flow rate (e.g., by opening or closing the measuring receptacle 110 openings to a certain degree, for example to improve incubation time for better measurements), modifying the carbon or material release rate, increasing/decreasing rate of data collection, etc.

In control feedback implementations, as mentioned above, the control system 140 may further control the carbon manipulation system 150. In these variations, the control system 140 may monitor the carbon concentration of the liquid, and then adjust the operation of the carbon manipulation system 150 to thereby increase or decrease the liquid carbon concentration.

The carbon manipulation system 150 functions to modify the water quality. The carbon manipulation system 150 in particular functions to modify the DIC concentration state through adding of a supplement. In one variation, the supplement may be an alkaline supplement such as an alkaline solution. In another variation, the supplement may be carbon dioxide gas or a gas that contains carbon dioxide (e.g., air, flue gas) that can be percolated or dispensed through the water. In some variations, pure (or concentrated CO₂ gas could be supplied by direct air capture and then the system may be used for carbon storage in water. Another option could be flue gas (lower concentration CO₂).

The carbon manipulation system 150 may be directly integrated with the measuring receptacle 110 such that the supplement is measured directly to water contained and enclosed by the measuring receptacle 110.

In another variation, the carbon manipulation system 150 may be in communication with the monitoring system and/or the control system 140. In this variation, the carbon manipulation system 150 may be removed and/or established separate from the monitoring system.

The carbon manipulation system 150 may include a supplement depositing mechanism to add solid or liquid supplement material to the water. The carbon manipulation system 150 may include a mixer or other system to integrate the supplement into water.

In some variations, the carbon manipulation system 150 may include a depositing mechanism that adds supplement in controlled and quantified amounts. In this way, the carbon manipulation system 150 may add a specified amount of supplement. In another variation, the carbon manipulation system 150 may include a depositing mechanism that adds in discrete amounts. This variation may add a discrete amount periodically until a target condition is satisfied.

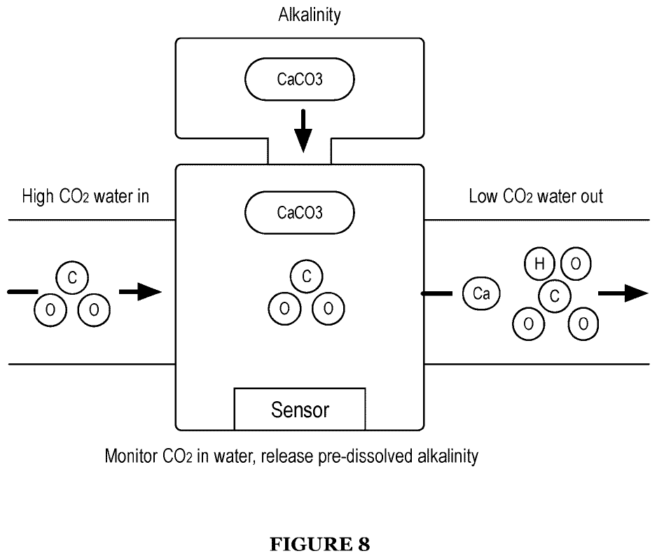

In one variation, the carbon manipulation system 150 is integrated between two monitoring systems as shown in exemplary FIG. 8. An initial upstream monitoring system may report on the base DIC concentration state, and the subsequent downstream monitoring system may report on the resulting DIC concentration state for an output. The sensors may be enabled to determine the pH, dissolved carbon content, and other chemistries of the fluid. In one implementation, the system may be implemented in proximity to a mining operation. With mining discharge, the sensors may detect high mineral contents, and potentially a high alkalinity and pH. The carbon manipulation system 150 can be controlled to dispense a supplement to alter the system.

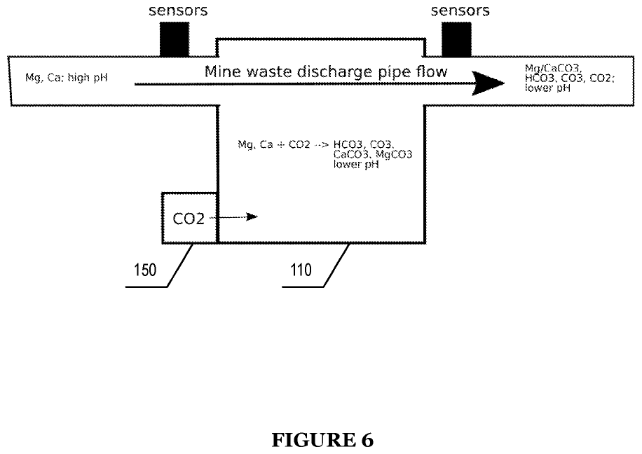

As shown in FIG. 6, water from an input may come with magnesium and calcium content and a high pH. The control system 140 may control the carbon manipulation system 150 to release carbon dioxide into the fluid, thereby increasing the DIC concentration and reducing the pH. As a generalized reaction, the magnesium and calcium can combine with the CO₂ within the water to produce HCO₃, CO₃, and a lower pH. The Ca and Mg would be dissolved ions in the water, balancing charge of HCO₃ and CO₃. The downstream sensor may be used for controlled feedback to fine tune the amount of carbon dioxide released in the fluid. The amount of CO₂ released may be adjusted based on measurements at the downstream monitoring system.

As shown in FIG. 8, water from an input with high CO₂ concentrations and low pH, may be modified with an alkaline compound such as CaCO₃. The control system 140 may control the carbon manipulation system 150 to release an alkaline supplement source. An alkaline supplement source can combine with the CO₂ within the water to form HCO₃ and a Calcium (e.g., in an aqueous, dissolved free ion, Ca²⁺), raising the pH in the process.

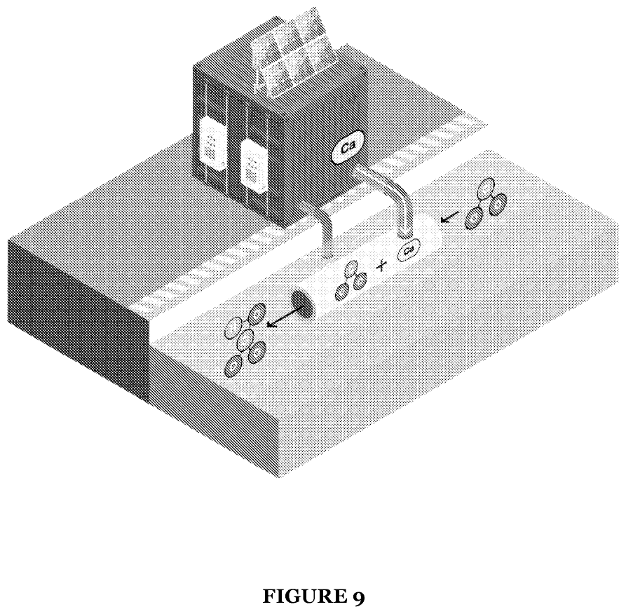

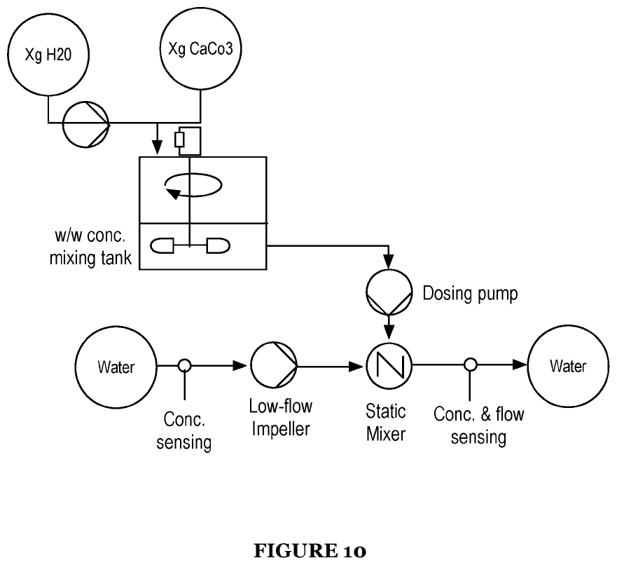

As shown FIG. 9, a carbon manipulation system 150 may be implemented as a station (docked on land or on floating or supported system over the water), that can facilitate mixing and adding of a supplement. As shown in FIG. 10, such a carbon manipulation system 150 may prepare a supplement and then add the supplement within the flow of water that is being measured and monitored. This approach may be used in natural water systems, but could also be used in closed water systems like in treatment of waste water from industrial applications.

### 3. Method

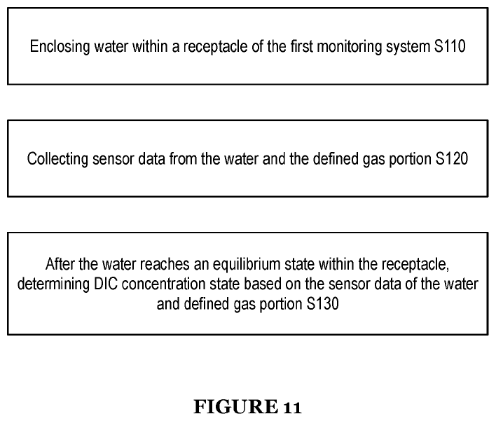

As shown in FIG. 11, a method for carbon monitoring may include at a first monitoring system, enclosing water within a receptacle of the first monitoring system, with the water at a filled level that leaves a defined gas portion within the receptacle S110; collecting sensor data from the water and the defined gas portion S120; after the water reaches an equilibrium state within the receptacle, determining DIC concentration state based on the sensor data of the water and defined gas portion S130.

The method functions to use a specially configured monitoring system that can established an isolated enclosed environment to measure and monitor DIC concentration state of a body of water. That is, the method leverages specific properties of the body of water and carbon concentration of a gaseous region in equilibrium with a portion of that body of water, to determine the carbon concentration of the body of water. With additional information regarding thermodynamic and molecular properties of some general, the method may be implemented to determine the carbon concentration of any fluid. The method implemented with a specially configured monitoring system may be implemented with a closed unmoving body of water (e.g., a lake), or an open dynamic body of water (e.g., river). The method may be used with the system as described herein but may alternatively be implemented in connection with alternative systems.

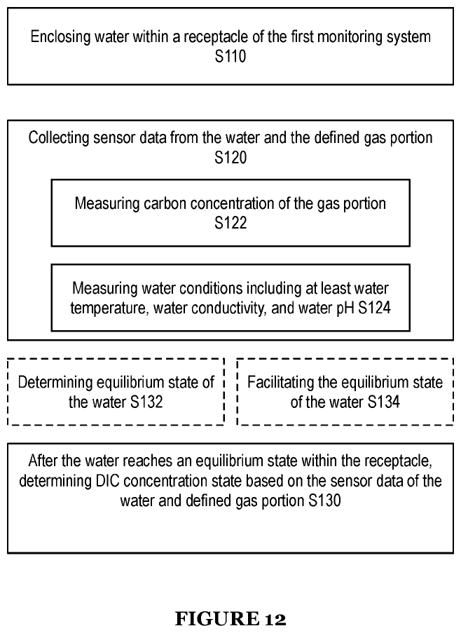

In some variations, collecting sensor data from the water and the defined gas portion S120 preferably collects sensor data concerning conditions of the water and the gas portion so as to determine condition of carbon-state conditions within the water. Block S120 may include measuring carbon concentration of the gas portion S122, and measuring water conditions including at least water temperature, conductivity, and water pH S124. Block S130 is particularly includes determining DIC concentration state based on carbon concentration, temperature, water conductivity, and water pH S130 as shown in FIG. 12. The resulting water carbon condition preferably includes characterization or measurements that include weight measurement of CO₂, HCO₃, and CO₃.

As part of managing monitoring of carbon condition, the method may further include determining equilibrium state of the water S132 and/or facilitating the equilibrium state of the water S134.

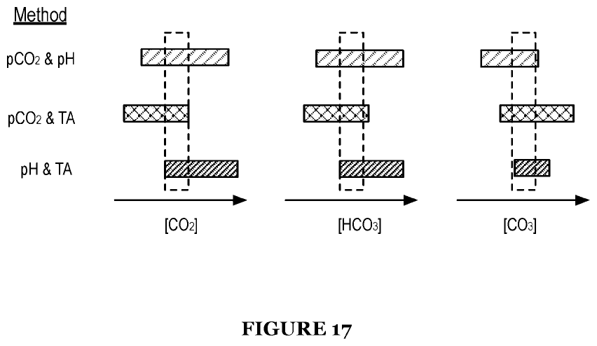

To determine the DIC concentration state (e.g., measurements of CO₂, HCO₃, and CO₃ in water), the method can depend on determining two constraining variables. i.e., pH and alkalinity or pH and CO₂ or alkalinity and CO₂, that can then be used as input to DIC modeling calculation methods to calculate the rest of the system. In the method, pH may be directly measured using a sensor. Alkalinity may not be available as a directly sensed value using affordable sensors-instead the method may apply an approach using electrical conductivity to quantify alkalinity (e.g., using a calibration and modeling approach that can account for salinity and temperature's effect on conductivity to quantity both salinity and alkalinity). Directly measuring CO₂ in water can also be practically challenging within a practical/affordable device. The method may use an approach that uses equilibrating the air and water, so that measuring CO₂ from air can be combined with data of salinity (from electrical conductivity) and temperature to calculate the solubility constant for CO₂ in water to calculate dissolved CO₂ in the water from measurements of CO₂ in the unfilled region. This approach may result in having 3 variables to calculate CO₂, HCO₃, and CO₃, while only two are needed to perform the necessary DIC modeling calculation. This approach thus enables three independent methods for DIC modeling calculations (pH & CO₂, pH & alkalinity, CO₂ and alkalinity), to then triangulate the various results from those three methods to get a refined, higher-accuracy measurement as shown in example FIG. 17.

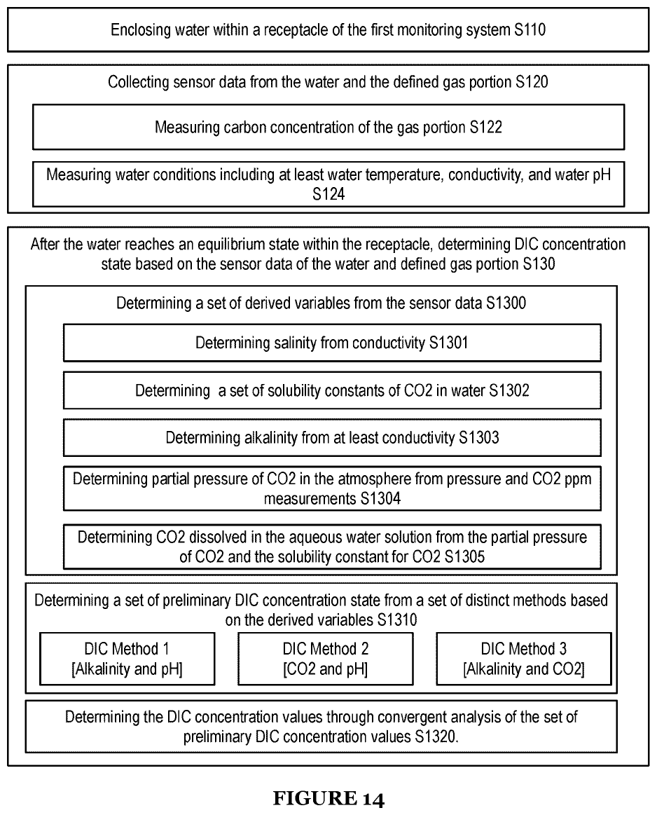

Accordingly in some variations, as shown in FIG. 14, the method may include at a first monitoring system, enclosing water within a receptacle of the first monitoring system, with the water at a filled level that leaves a defined gas portion within the receptacle S110; collecting sensor data from the water and the defined gas portion S120; after the water reaches an equilibrium state within the receptacle, determining DIC concentration state based on the sensor data of the water and defined gas portion S130, which includes determining a set of derived variables from the sensor data S1300 including salinity, a set of solubility constants of CO₂ in the water, alkalinity, partial pressure of CO₂ and CO₂ dissolved in the aqueous water solution; determining a set of preliminary DIC concentration states from a set of distinct DIC modeling methods based on the derived variables S1310; and determining the DIC concentration values through convergent analysis of the set of preliminary DIC concentration states S1320.

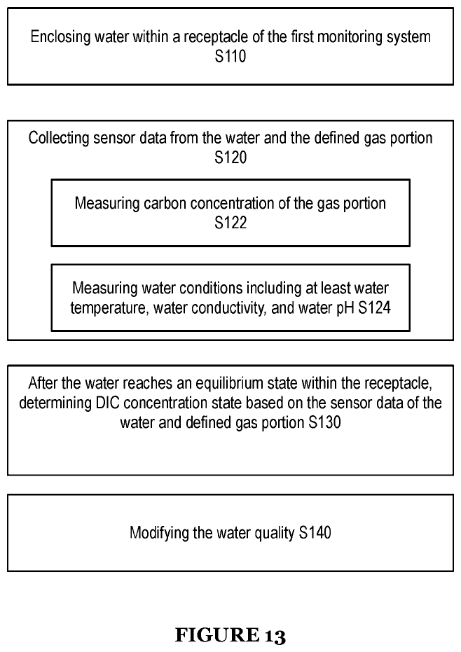

Some method variations may further be extended for carbon management of the fluid, wherein carbon-state may be directly impacted and/or augmented. In these variations, the method may further include: modifying the water quality S140, which may be performed based on and/or in response to the DIC concentration as shown in FIG. 13. In such water management method variations, the method may function to actively monitor and control the water quality. In addition to monitoring and modifying the DIC concentration, the method may control water quality by modifying water pH, water alkalinity, water mineral content, and/or water mineral quality (e.g., by the formation of salts). The modification of the water quality can be controlled and regulated using control feedback from the determined DIC concentrations of the water.

In some variations, the method may be implemented in connection with a single monitoring system. In other variations, the method may be implemented in connection with multiple monitoring systems that are used in combination for monitoring carbon state of a water system.

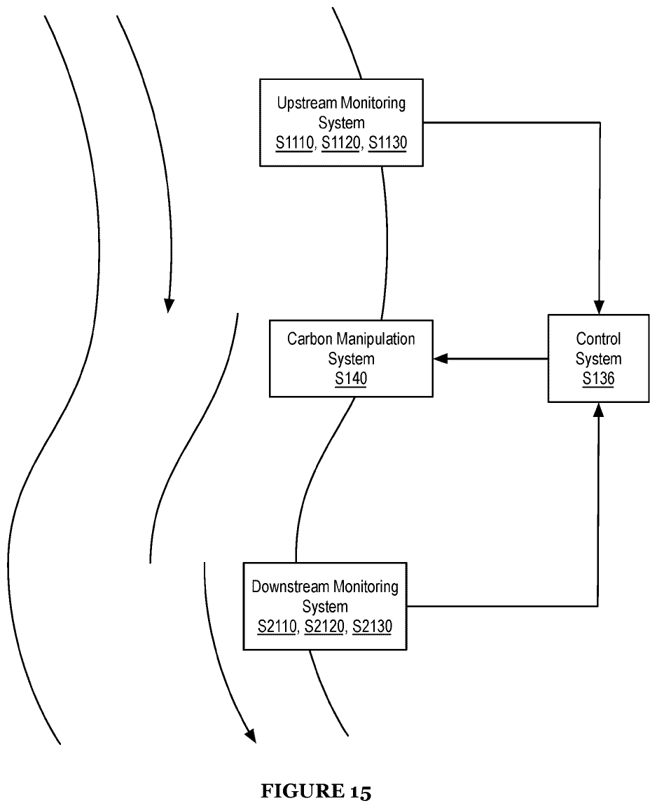

As shown in FIG. 15, a method for carbon monitoring within a network may include at a first monitoring system, enclosing a first volume of water within a first receptacle of the first monitoring system, with the water at a filled level that leaves a defined gas portion within the first receptacle S1110; collecting sensor data from the water and the defined gas portion S1120; after the water reaches an equilibrium state within the first receptacle, determining DIC concentration state of the first monitoring system based on the carbon concentration, temperature, water conductivity, and water pH S1130; and at a second monitoring system, enclosing a second volume of water within a second receptacle of the second monitoring system, with the second volume of water S2110, with the water at a filled level that leaves a defined gas portion within the second receptacle (i.e. the second defined gas portion), wherein the second monitoring system is downstream from the first monitoring system within a water system; collecting sensor data (e.g., a second set of sensor data) from the second volume of water and the defined gas portion within the second receptacle S2120; after the water of the second monitoring system reaches an equilibrium state within the second receptacle, determining DIC concentration state at the second monitoring system S2130 based on the carbon concentration, temperature, water conductivity, and water pH; and comparing DIC concentration state at the first monitoring system to the second monitoring system and determining a change in DIC concentration states S136.

The two monitoring systems can preferably individually implement the monitoring operations described herein, and then results may be compared and/or used in combination. For example, collecting sensor data (e.g., a second set of sensor data) from the second volume of water and the defined gas portion within the second receptacle S2120 may include measuring carbon concentration of the gas portion of the second monitoring system, and measuring water conditions of the second monitoring system including at least water temperature, water conductivity, and water pH.

In one exemplary application, a two or more monitoring systems may be used within an industrial wastewater system where an initial upstream monitoring system is used for monitoring initial DIC concentration states and a subsequent downstream monitoring system is used for monitoring resulting DIC concentration states. This may be used for treating water resulting from an industrial process. This may also be used for monitoring impact of an industrial process on water source.

In another exemplary application, two or more monitoring systems may be used within a natural water system. For example, an initial upstream monitoring system may be situated at an upstream location within a flowing water system, and a subsequent downstream monitoring system may be situated at a location downstream in the flowing water system.

The method may similarly be used across a large network with several monitoring points, and/or several points or regions where modification of the water system may be applied.

Block S110, which includes enclosing water within a receptacle of the first monitoring system, functions to acquire a sample of a body of water for analysis. Enclosing water within the receptacle can include filling the receptacle. The receptacle is preferably partially filled such that part of the receptacle is filled with water (i.e., the filled region or water portion) and part of the receptacle is empty (i.e., the unfilled region or gas portion). Accordingly, the water is enclosed at a filled level that leaves a defined gas portion within the receptacle. The monitoring system preferably includes a receptacle that is equipped or otherwise configured for sensing of water and gas conditions.

In some variations, block S110 is a single operation, wherein a single sample is taken. Alternatively, block S110 may comprise setting up the detection receptacle such that water continuously travels through the filled region, whereas the gas portion stays empty (of water/liquid).

Enclosing the water within the receptacle of the first monitoring system can include receiving water from an external water system through an inlet of the monitoring system/receptacle until the water reaches the filled level. After the water reaches the equilibrium state and/or sensing and monitoring is completed, the method may include releasing the water.

In some variations, the external water system is a natural water system. A natural water system could include flowing natural water systems like streams, rivers, tidal estuaries, and coastal waters, but may also include substantially stagnant water systems like ponds, lakes, reservoirs. In some variations, the external water system is an industrial water system such as a wastewater system.

In some variations, water is received from a water source and then the monitored water may be released back into that same water source. In some variations, water may be received from a water source and then released to some water output. This may be particularly used in industrial applications where water from some source is received via an inbound conduit (e.g., pipe, channel, etc.) and then released to some outbound conduit.

In one variation, the monitoring system may include a single water opening. The water opening may be dynamically opened and closed, such that water may be allowed to enter exist the receptacle. Accordingly, enclosing the water may include opening a receptacle opening thereby allowing water to fill the receptacle and closing the receptacle opening after completion thereby allowing the water to exit the receptacle. In some variations, the monitoring system may additionally include a controllable air valve 126 that can mechanically open and close to allow air in for draining of water and subsequently closing off when the receptacle is refilled. Accordingly, enclosing the water may include opening a receptacle opening and an air valve thereby allowing water to fill the receptacle and closing the receptacle opening and the air valve after completion thereby allowing the water to exit the receptacle. In some cases, receptacle is not emptied, but new water is allowed to flow in and displace the previously contained water.

In another variation, the monitoring may include an inlet and outlet. The inlet may be opened to allow water in and closed when new water reaches the filled level. The outlet may be opened to allow water to leave, and then closed facilitate enclosing a volume of water.

In some variations, the method may include detecting the water reaching the filled level. Detecting the water level in one variation can include using a float switch that uses a buoyant float to trigger a change in an electrical signal when the water rises to a certain level. Detecting the water level may alternatively include using an optical/visual depth sensor, using conductive water level sensor on the edge of the receptacle, and/or other sensing approaches. In some variations, the method may not depend on achieving one specific volume of water. The method may include sensing water level, which may be used to calculate the volume of water and/or gas portion. Water and gas volume variables may be factored into process of determining DIC concentration states. Other alternative methods may manage the fill level using non-sensing approaches. In one such variation, the amount of water added may be controlled, regulated, or enforced. In one exemplary variation, the fill level may be controlled or managed by controlling flow of water into the container such that the fill level may be determined by the amount of time water was added to the receptacle.

In a continuous flow variation, the rate of inflow and outflow of water from the system could be the same such that the water and air volumes within the container remain the same, and changes in the carbon of the water can be quantified in a continuous flow fashion. The volume of both air and water can be optimized to detect more rapid or larger magnitude variability in the water carbon.

Block S120, which includes collecting sensor data from the water and the defined gas portion functions to detect a set of measurable parameters of the water and gas portions within the receptacle. These measurable parameters can be used in evaluating state of various chemical reactions that can indicate DIC concentration states.

In particular, collecting sensor data preferably collects sensor data for water temperature, water conductivity, water pH, pressure in the gas portion, and detecting CO₂ concentrations in the gas portion. Accordingly, collecting sensor data from the water and the defined gas portion may include measuring and/or sensing at least carbon concentration of the gas portion S122 and measuring and/or sensing water conditions including at least water temperature, conductivity, and water pH S124. The collected sensor data is preferably collected from a set of different sensors integrated within the enclosure. The sensor data can be communicated or otherwise transferred to a computing device.

Some sensor data measurements are monitored and read periodically as a way for assessing and/or determining an equilibrium state. For example, CO₂ and pH may be periodically monitored for a steady-state condition indicating an equilibrium state of the water. In one exemplary implementation, these measurements may be made periodically (e.g., every 3 seconds), though any suitable time period may be used. The time period may additionally be adjusted based on the volume of water within the receptacle. The periodic measurements can be made during filling and after until an equilibrium state is determined. The equilibrium state may be determined when CO₂ does not change by more than some threshold (e.g., the precision of the sensor used) for a configured amount of time (e.g., 30 seconds). Then all measurement parameters may be taken at the final “at equilibrium” point for calculating the DIC component state of the water. Other sensor data inputs may be sensed during the equilibrium state so that they may be used in determining the DIC concentration state.

In some variations, measurements may be performed periodically (e.g., every 10 seconds) providing substantially continuous record of both baseline metrics, and in the case where carbon dioxide removal (CDR) practices are implemented metrics qualifying impact of CDR. These data, as well as system status and performance metrics, may automatically uploaded to an online dashboard for remote monitoring by operators, third-party MRV providers, regulatory agencies, and any other interested parties.

Block S122, which includes measuring carbon concentration of the gas portion, functions to determine the CO₂ level within the gas portion. Measuring carbon concentration preferably measures the concentration of CO₂ in the gas portion, though additional or alternative carbon compounds may be sensed as well. Sensing CO₂ or other carbon-based molecules may use a broad range of spectrometers. As the primary forms of carbon include carbon dioxide, a spectrometer functioning in the infrared spectrum may be sufficient to measure the carbon concentration of the unfilled region. Measuring CO₂, in some variations, may use a spectroscopic sensor like an NDIR sensor.

In addition to measuring carbon concentration of the gas portion, collecting sensor data may include measuring pressure within the gas portion. Accordingly in some variations an alternative process to block S122 can include measuring carbon concentration and pressure of the gas portion. In one variation, measuring pressure may use a gas pressure sensor that is integrated into the receptacle and oriented to sense within the gas portion. Alternative variations may estimate or use alternative techniques to incorporating pressure. In one variation, pressure may be held static with a responsive pressure valve, which functions to make the pressure a known amount within the gas portion. Knowing pressure improves accuracy for converting from air CO₂ to water CO₂ by better constraining the dissolution constant for CO₂.

Block S124, which includes measuring water conditions, functions to assess a set of parameters used to determine state of the water. Measuring water conditions can include measuring at least water temperature, water conductivity, and/or water pH. Water salinity and/or alkalinity are also other parameters of interest that may be derived using sensed data. For example salinity may be based in part on temperature and the electrical conductivity of the water; and alkalinity may be based in part on the electrical conductivity and other parameters depending on the implementation. Measuring the water properties S134 may occur within the detection receptacle, but particularly in the case of large body of waters, block S134 may be implemented anywhere in the body of water that would have similar properties of the acquired sample.

Block S130, which includes determining DIC concentration state based on the carbon concentration, temperature, water salinity, and water pH, functions to use the measurable properties after the water reaches an equilibrium state within the receptacle to deduce the levels of various carbon compounds using assumed chemical reactions. At steady state, the gaseous carbon concentration may be used in conjunction with the properties of the water to calculate the DIC concentration.

Dependent on implementation, block S130 may further determine the concentration of one or more different molecular forms of carbon. In one variation, the water carbon condition may be characterized by at least measurements (e.g., concentration measurements) of at least CO₂, HCO₃, and CO₃. That is, determining the DIC concentration S130 may further include determining the water carbon dioxide concentration, determining the water carbonate concentration, and/or determining the water bicarbonate concentration.

Measuring the DIC concentration states preferably uses the collected sensor data from block S120 from the gas portion and the water portion to determine several intermediary parameters and then determine measurements of individual DIC components: CO₂, HCO₃, and CO₃. Accordingly, in one variation, determining the DIC concentration states can include: determining a set of derived variables from the sensor data S1300, determining a set of preliminary DIC concentration state from a set of distinct methods (i.e., DIC modeling methods) based on the derived variables S1310, and determining the DIC concentration values through convergent analysis of the set of preliminary DIC concentration values S1320 as shown in FIG. 14. Using multiple methods for deriving the DIC concentration state can function to improve the prediction of the DIC concentration state by triangulating from different methods with different biases in their modeling. Different methods can rely on different relationships and/or chemical transformations/reactions for modeling predicted DIC concentration values. The convergent analysis can assess these different predicted DIC concentration values in combination to output a preferably more reliable and accurate prediction of DIC concentration state. Because the resulting reactions can be highly multivariant, using convergent analysis of different distinct methods can result in a more accurate prediction that is less vulnerable to the assumptions of an individual method.

Additionally, the final DIC concentration state cannot only characterize measurements for the combination of DIC components, but can provide individual measurements for multiple DIC components, namely CO₂, HCO₃ and CO₃²⁻.

In some variations however, the method may be simplified to using a single method where determining the DIC concentration states includes determining a set of derived variables from the sensor data and then directly determining the DIC concentration state from the set of derived variables. This variation may use one DIC modeling method. This may be used in certain situations or implementations. For example, in some applications, the initial state of the water may be substantially stable such that one method may be to a satisfactory job of predicting measurements of the DIC concentration state.

Block S1300, which includes determining a set of derived variables from the sensor data, functions to use the sensor data to determine various other intermediate parameters usable for assessing the DIC concentration state. The exact derived variables that are determined may depend on the method(s) used for modeling DIC concentration states.

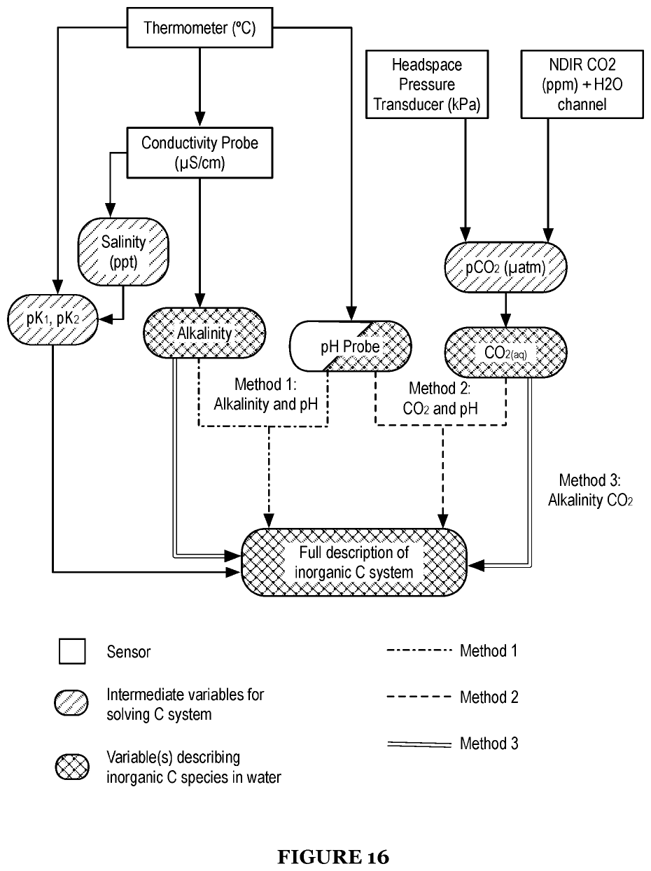

As shown in FIGS. 14 and 16, determining the set of derived variables from the sensor data may include initially determining salinity from conductivity S1301, determining a set of solubility constants of CO₂ in water, including, pK1, and pK2, from temperature and salinity and pressure S1302, determining alkalinity from at least conductivity S1303, determining partial pressure of CO₂ (e.g., μatm) in the atmosphere from pressure and CO₂ ppm measurements S1304, and determining CO₂ dissolved in the aqueous water solution from the partial pressure of CO₂ and the solubility constant for CO₂, K_{CO2} S1305. The DIC components (or species) of CO₂, HCO₃⁻ and CO₃²⁻ can be represented through equations:

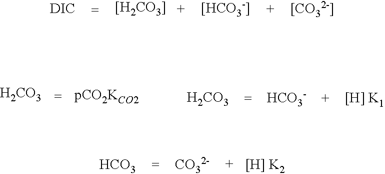

Dissolved CO₂ can include two pools, free CO₂ and H₂CO₃. K_{co2}, the solubility coefficient for CO₂ from air to water, and K1 and K2, the first and second dissociation constants of carbonic acid (H₂CO₃), are functions of salinity and temperature. pCO₂ is the partial pressure of CO₂ gas in the atmosphere at equilibrium with the water.

Determining salinity from conductivity S1301 can use an electrical conductivity measurement of the water in combination with temperature measurements to predict salinity. The salinity may be measured in ppt.

Determining the set of solubility constants of CO₂ in water from temperature, salinity and pressure S1302 can determine solubility constants K_{CO2}, pK1, and pK2.

Determining alkalinity from conductivity S1303 can use conductivity in combination with other sensor data and/or intermediate derived variables. The alkalinity may be measured in relevant units like micromol/kg. Alkalinity in one variation may be derived based on a combined analysis of pH, aqueous CO₂ concentration and the conductivity, or may be derived by only the conductivity when incorporated into numerical models that can be used to directly quantify alkalinity. Accounting for the temperature-dependence, electrical conductivity may be interpreted or modeled as a statement about the bulk composition of a sample-salinity, total dissolved solids (TDS), alkalinity, and ionic strength. In some modeling approaches alkalinity can be expressed as a linear relationship between conductivity and alkalinity, using both empirically-derived regressions and/or theoretically-derived models that account for the ionic strength and activity of individual ions. Dynamic calibration of this linear modeling can further enhance this approach.

In some variations, a machine learning model may be trained on predicting alkalinity based on pH, conductivity and CO₂ concentrations. Accordingly, determining alkalinity may include processing conductivity, pH, CO₂ concentrations and/or other additional sensor data and/or derived variables within a model (e.g., a trained AI/ML model like a neural network) and outputting an alkalinity measurement as a derived variable. In another variation, derived CO₂ may be used with other sensor data and/or derived variables and one DIC modeling method to determine a prediction of preliminary DIC concentration state using that method, and then applying results to back calculate alkalinity measurement that can be used within one or more other DIC modeling methods in S1310.

Determining partial pressure of CO₂ in the atmosphere from pressure and CO₂ ppm measurements S1304 functions to use pressure measurements from the gas portion (e.g., the headspace pressure measured using a pressure transducer) and CO₂ concentration measurements (e.g., using an NDIR sensor measuring CO₂ in ppm).

Determining CO₂ dissolved in the aqueous water solution from partial pressure of CO₂ and the solubility constant for CO₂ Ko S1305 functions to use solubility constant of CO₂ and the partial pressure of CO₂ in air above the water, at equilibrium, to predict CO₂ concentration in the water. Both the volumes of water and air must be known, and equilibrium must be established. In some variations, the water may be distributed using a sprayer or some other mechanism to perturb the water. Additionally sensor data may be monitored to detect when equilibrium is established based on settling of sensor data (e.g., reaching steady state).

Block S1310, which includes determining a set of preliminary DIC concentration state from a set of distinct methods based on the derived variables, functions to use different independent DIC modeling methods for determining measurements of the DIC components. Each method can generate one preliminary DIC concentration state measurement. In one variation, there can be three different DIC modeling methods used. However, some variations may use two or more than three different DIC modeling methods. In one variation, a first DIC modeling method includes predicting a first preliminary DIC concentration state based on modeling of alkalinity and pH; a second DIC modeling method can include predicting a second preliminary DIC concentration state based on modeling of CO₂ and pH; and a third DIC modeling method may include modeling a third DIC concentration state based on modeling of alkalinity and CO₂. The different methods, as in the exemplary variations of the first, second and third DIC modeling methods above, can use different inputs and may be based on different modeling assumptions or calculations.

The first DIC modeling method may use Alkalinity and pH for deriving one preliminary DIC concentration state. For example, CO₂ can be independently calculated using=(alkalinity−((Kb (the boron solubility constant)*Bt(concentration of boron))/(Kb+H))−((10\hat{ }−7)/H)+H)/((K1/H)+(2*((K1*K2)/(H\hat{ }2)))), which can then be used as above for calculating DIC and other parameters.

The second DIC modeling method can use CO₂ and pH for deriving the different DIC component measurements to establish one preliminary DIC concentration state. Once the dissolved CO₂ has been calculated using the above, it can be used to calculate the total concentration of all of the dissolved inorganic carbon ions (CO₂, HCO₃, CO₃) using the equation: DIC=CO₂*(1+(K1/h)+((K1*K2)/h2)) where h is the standard hydrogen ion concentration (derived from pH) and CO₂ is in μatm. Once total DIC is calculated from the above, that known quantity, pH, and CO₂ can be used to calculate HCO₃ and CO₃ and other relevant parameters (e.g., carbonate saturation state). As discussed above, sometimes this method based on CO₂ and pH may be used to infer or determine an alkalinity measurement that can be used in other methods. However, different approaches for determining alkalinity may alternatively be used, including the electrical conductivity approach described above.

The third DIC modeling method can use alkalinity and CO₂ to determine another preliminary DIC concentration state. In this method, alkalinity and CO₂ are used in a fourth-order equation to calculate pH, which is then incorporated into the above method for the calculation of the remaining DIC compounds.

Block S1320, which includes determining the DIC concentration values through convergent analysis of the set of preliminary DIC concentration values, functions to triangulate or otherwise perform combined analysis of the different modeled DIC concentration states to determine some combined output. For example, the different DIC modeling methods may generate a first set of DIC concentration state measurements for CO₂, HCO₃ and CO₃, a second set of DIC concentration state measurements for CO₂, HCO₃ and CO₃, and a third set of DIC concentration state measurements for CO₂, HCO₃ and CO₃. The convergent analysis can look at the overlap of value predictions of the different measurements to determine a final DIC concentration state as shown in FIG. 17. In another, the convergent analysis may use some averaging (e.g., weighted average) of different DIC concentration values.

Determination of the DIC concentration state can rely on the water achieving an equilibrium state within the enclosed receptacle. Accordingly, the method may include block S132 determining or detecting equilibrium state of the water, which functions to identify when the system (e.g., interactions between water and the gas) has relaxed to a steady state. That is, the system is allowed to sit until there is no net molecular exchange between the filled and unfilled regions of the detection receptacle. For the system to reach this steady state, it may be required to seal the unfilled region (and possibly the filled region).

In some variations, the determination may be based on a prediction using various factors. In this way determining the equilibrium state may include predicting an equilibrium state. For example, the equilibrium state may be predicted to occur after some time period. In this way, water could be enclosed, allowed to sit for some amount of time, and then the DIC concentration state analyzed. The amount of time may be variable based on various conditions such as initial temperature, pressure, pH, CO₂ levels. Accordingly, initial sensor data may be measured when predicting the equilibrium state.

In other variations, the determination of the equilibrium state may be actively detected using sensor data. For example, steady state of CO₂ and/or pH may be indicators of an equilibrium state. In this way determining the equilibrium state of the water may include detecting equilibrium state by measuring CO₂ and pH periodically for an enclosed volume of water. The CO₂ and pH measurements can be monitored until they reach a steady-state condition. The steady-state condition is generally when the CO₂ and PH levels don't change anymore. The sensitivity and/or error thresholds of the sensors and acceptable levels of fluctuation may be incorporated into the condition. For example, CO₂ may be monitored for a consistent reading with less than 5 ppm of variance which corresponds to the error range of some sensors.

For a continuous flow implementation, the system may be prevented from reaching a steady state, as the unfilled region would be continuously replenished from the exterior environment.

Thus, for a continuous flow implementation of the filled region, the method may use a body of water for measurement that is sufficiently large that it may act as a reservoir, such that particulate concentration of the body of water is unaffected by reaching a steady state with the unfilled region of the detection receptacle.

The time required for reaching a steady state may be implementation specific. Dependent on external environmental conditions (e.g., temperature, carbon disparity between air and water, etc.), the time required for equilibrating the filled region and the unfilled region S120 may vary. In some new implementations, this time may be optimized by making an initial sample acquisition and monitoring the carbon fluctuations in the unfilled region. This may be done multiple times at different temperatures, to potentially determine the time required at different times of day.

Block S134, which includes facilitating the equilibrium state of the water, functions to alter the establishment of an equilibrium state. In one variation, facilitating the equilibrium state can include spraying, aerating, shaving, mixing, or vaporizing water or otherwise disturbing the water enclosed within the receptacle. In some variations, this may function to ensure an equilibrium state when taking measurements for determining the DIC concentration state.

As discussed, some variations may implement multiple monitoring systems such that monitoring of the DIC concentration states may be tracked at different locations and/or stages of water alteration (e.g., from industrial practices, modification via S140, etc.).

Implementations with multiple monitoring systems can have each monitoring system performing the monitoring processes described herein. In some variations, there may be two monitoring systems within a flowing water system with one monitoring system used upstream and second monitoring system used downstream. In other variations, monitoring systems may be distributed across a region in multiple regions. For example, if monitoring a large body of water like a lake, then monitoring systems may be distributed at various points to establish a “map” of DIC concentration states.

When multiple DIC concentration states are measured, then the method may include comparing DIC concentration state at the first monitoring system to the second monitoring system and determining a change in DIC concentration states S136. Block S136 functions to assess altered state between one monitoring system and another monitoring system. When used within a flowing water system, this may be used to see how water changed. In some variations, the first monitoring system may be oriented upstream to determine initial conditions, then between the first and second monitoring systems, some augmentation or change may be introduced to the water, then after that change a subsequent downstream monitoring system can determining the measuring carbon concentration conditions resulting from that change. Comparing the results from the two monitoring systems may be used to determine the impact of the change.

In some variations, the introduced change may be natural like a stream flowing into a main river system. In some variations, the introduced change may be from some industrial process or system.

In one variation, the introduced change may be introduction of supplements based on the DIC concentration states as part of S140.

Block S140, which includes modifying the water quality, functions to alter the water composition, by altering the water pH, water alkalinity, and/or DIC concentration. Modifying the water quality S140, may be in response to the measured DIC concentration, or other water qualities, as measured by block S130.

One variation for modifying water quality S140 includes adding a supplement and regulating the adding of the supplement to satisfy a target water carbon condition. In some variations where the CO₂ is elevated in the water, the supplement is an alkaline, which may be supplied and added in a variety of forms. In one variation, the alkaline supplement is supplied through a solution and mixed with the water. The target DIC concentration state could be targeted by adding an alkaline supplement such that the pH is kept less than 8.5, or raised higher for precipitation and management of solid carbon minerals.

In some other variations, the supplement could be CO₂ such as captured gaseous CO₂, flue gas, or ambient air. Modifying water quality S140 can include dispensing gaseous CO₂ through the water. This may additionally include regulating the dispensing of the gaseous dioxide to satisfy a target water carbon condition. This may be done, for example, in water systems that have amounts of magnesium and calcium as well as a high pH. Introduction of the CO₂ can result in reactions that convert the magnesium, calcium, and CO₂ to HCO₃, CO₃, CaCO₃, MgCO₃. The pH of the water may additionally be lowered through such reactions.

When there are multiple monitoring systems, they may be used to determine a change in DIC concentration states between the two monitoring systems. In this scenario, modifying the water quality may be based on this change. Accordingly, modifying the water quality, in one variation, includes adding a supplement or additive to the water system and regulating the adding of supplement based on the determined change in DIC concentration states. The supplement may be a dry powder or concentrate that is added to the water. In other variations, the supplement may be a dilution. Adding the supplement may additionally include processes to integrate to initiate reactions or integration of the supplement with the water. For example, adding the supplement may include percolating a supplement gas through the water or in another example stirring or mixing a solution within water.

Different supplements or actions may be taken for different conditions and/or implementations. In one exemplary scenario variation, where the local water is highly alkaline and mineral rich, modifying the water quality S140 may work to reduce the pH (e.g., by adding carbon dioxide). Adding CO₂ may then cause converting CO₂ to HCO₃ and CO₃ (e.g., for storage).

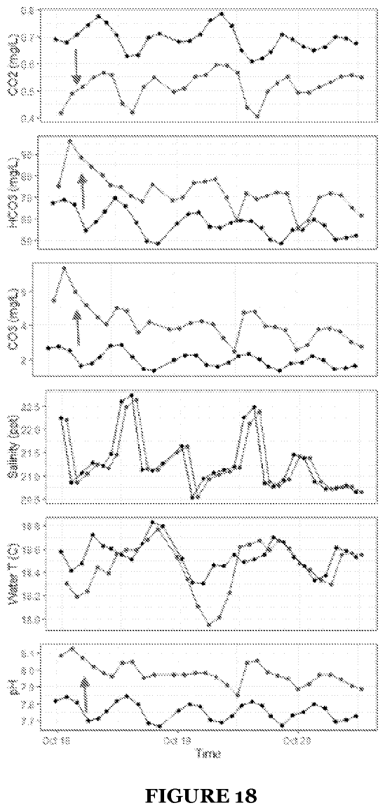

In another scenario variation, wherein the carbon concentration of the water is high and/or the water has a low pH, modifying the water quality S140 may add alkaline compounds for increasing the pH, capturing/removing carbon from the water. Alkaline compounds may be introduced to initial carbon chemistry of the water (CO₂, HCO₃, CO₃ concentration and pH) to cut down on excess CO₂ and have it leave the system with CO₂ at equilibrium with the atmosphere (e.g., 425 ppm) and with pH below a threshold (e.g., less than 8.5. As shown in FIG. 18), adding alkalinity may be used to result in decrease of CO₂, increase in HCO₃ and CO₃, and in keeping pH less than 8.5. The alkalinity may convert dissolved CO₂ to HCO₃ and CO₃ via a generalized pathway of CO₂(aq)+H₂O+CaCO₃ (or other alkalinity)→2HCO₃(aq)+Ca2+(aq).

Modifying the water quality by adding a supplement can include adding the supplement within the receptacle but may alternatively include adding external to the receptacle. In some variations, an external carbon manipulation system. The carbon manipulation can be controlled to deposit a supplement. In some variations, it may add discrete amounts at different times. In other variations, variable amounts of a supplement may be added. In some variations, the amount of supplement that is added is based on current state of the DIC concentration states. Adding the supplement may additionally include mixing, dispersing, or otherwise integrating the supplement within the water system. The modification may be performed until a desired condition is met. In some variations, this may be done through iterative steps of adding a supplement, evaluating new DIC concentration states after equilibrium of a water sample, and then repeating until the DIC concentration states satisfy match a targeted condition.

### 4. System Architecture

The systems and methods of the embodiments can be embodied and/or implemented at least in part as a machine configured to receive a computer-readable medium storing computer-readable instructions. The instructions can be executed by computer-executable components integrated with the application, applet, host, server, network, website, communication service, communication interface, hardware/firmware/software elements of a user computer or mobile device, wristband, smartphone, or any suitable combination thereof. Other systems and methods of the embodiment can be embodied and/or implemented at least in part as a machine configured to receive a computer-readable medium storing computer-readable instructions. The instructions can be executed by computer-executable components integrated with apparatuses and networks of the type described above. The computer-readable medium can be stored on any suitable computer readable media such as RAMs, ROMs, flash memory, EEPROMs, optical devices (CD or DVD), hard drives, floppy drives, or any suitable device. The computer-executable component can be a processor, but any suitable dedicated hardware device can (alternatively or additionally) execute the instructions.

In one variation, a system comprising of one or more computer-readable mediums (e.g., non-transitory computer-readable mediums) storing instructions that, when executed by the one or more computer processors, cause a computing platform to perform operations comprising those of the system or method described herein such as: enclosing water within a receptacle of the first monitoring system, with the water at a filled level that leaves a defined gas portion within the receptacle; collecting sensor data from the water and the defined gas portion; after the water reaches an equilibrium state within the receptacle, determining DIC concentration state based on the sensor data of the water and defined gas portion.

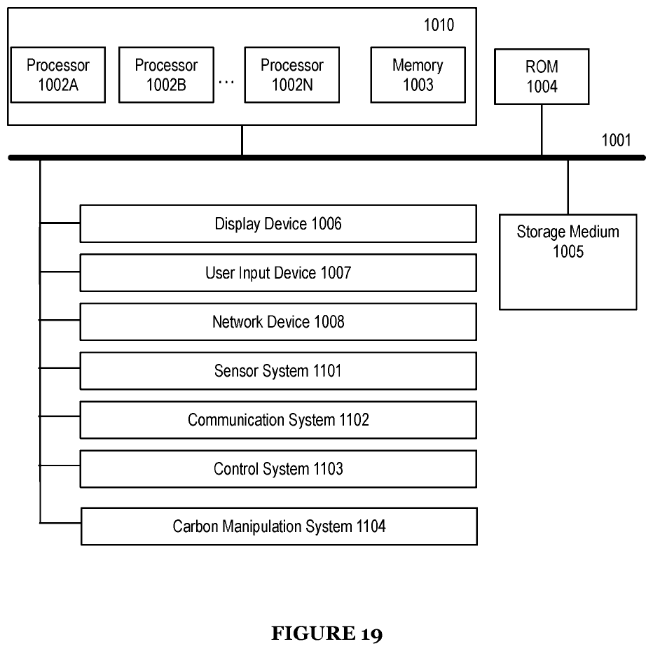

FIG. 19 is an exemplary computer architecture diagram of one implementation of the system. In some implementations, the system is implemented in a plurality of devices in communication over a communication channel and/or network. In some implementations, the elements of the system are implemented in separate computing devices. In some implementations, two or more of the system elements are implemented in same devices. The system and portions of the system may be integrated into a computing device or system that can serve as or within the system.

The communication channel 1001 interfaces with the processors 1002A-1002N, the memory (e.g., a random access memory (RAM)) 1003, a read only memory (ROM) 1004, a processor-readable storage medium 1005, a display device 1006, a user input device 1007, and a network device 1008. As shown, the computer infrastructure may be used in connecting sensor system 1101, communication system 1102, control system 1103, carbon manipulation system 1104, and/or other suitable computing devices.

The processors 1002A-1002N may take many forms, such CPUs (Central Processing Units), GPUs (Graphical Processing Units), microprocessors, ML/DL (Machine Learning/Deep Learning) processing units such as a Tensor Processing Unit, FPGA (Field Programmable Gate Arrays, custom processors, and/or any suitable type of processor.

The processors 1002A-1002N and the main memory 1003 (or some sub-combination) can form a processing unit 1010. In some embodiments, the processing unit includes one or more processors communicatively coupled to one or more of a RAM, ROM, and machine-readable storage medium; the one or more processors of the processing unit receive instructions stored by the one or more of a RAM, ROM, and machine-readable storage medium via a bus; and the one or more processors execute the received instructions. In some embodiments, the processing unit is an ASIC (Application-Specific Integrated Circuit). In some embodiments, the processing unit is a SoC (System-on-Chip). In some embodiments, the processing unit includes one or more of the elements of the system.

A network device 1008 may provide one or more wired or wireless interfaces for exchanging data and commands between the system and/or other devices, such as devices of external systems. Such wired and wireless interfaces include, for example, a universal serial bus (USB) interface, Bluetooth interface, Wi-Fi interface, Ethernet interface, near field communication (NFC) interface, and the like.

Computer and/or Machine-readable executable instructions comprising of configuration for software programs (such as an operating system, application programs, and device drivers) can be stored in the memory 1003 from the processor-readable storage medium 1005, the ROM 1004 or any other data storage system.

When executed by one or more computer processors, the respective machine-executable instructions may be accessed by at least one of processors 1002A-1002N (of a processing unit 1010) via the communication channel 1001, and then executed by at least one of processors 1001A-1001N. Data, databases, data records or other stored forms data created or used by the software programs can also be stored in the memory 1003, and such data is accessed by at least one of processors 1002A-1002N during execution of the machine-executable instructions of the software programs.

The processor-readable storage medium 1005 is one of (or a combination of two or more of) a hard drive, a flash drive, a DVD, a CD, an optical disk, a floppy disk, a flash storage, a solid state drive, a ROM, an EEPROM, an electronic circuit, a semiconductor memory device, and the like. The processor-readable storage medium 1005 can include an operating system, software programs, device drivers, and/or other suitable sub-systems or software.

As used herein, first, second, third, etc. are used to characterize and distinguish various elements, components, regions, layers and/or sections. These elements, components, regions, layers and/or sections should not be limited by these terms. Use of numerical terms may be used to distinguish one element, component, region, layer and/or section from another element, component, region, layer and/or section. Use of such numerical terms does not imply a sequence or order unless clearly indicated by the context. Such numerical references may be used interchangeable without departing from the teaching of the embodiments and variations herein.

As a person skilled in the art will recognize from the previous detailed description and from the figures and claims, modifications and changes can be made to the embodiments of the invention without departing from the scope of this invention as defined in the following claims.

We claim:

1. A method comprising:
at a first monitoring system, enclosing water within a receptacle of the first monitoring system, with the water at a filled level that leaves a defined gas portion within the receptacle;
collecting sensor data from the water and the defined gas portion comprising:
measuring carbon concentration of the gas portion, and
measuring water conditions including at least water temperature, water conductivity, and water pH;
after the water reaches an equilibrium state within the receptacle, determining dissolved inorganic carbon concentration state based on the carbon concentration, temperature, water conductivity, and water pH.

2. The method of claim 1, wherein enclosing the water within the receptacle of the first monitoring system comprises, receiving water from an external water system through an inlet of the receptacle until the water reaches the filled level; and, after the water reaches the equilibrium state, releasing the water.

3. The method of claim 2, wherein the external water system is a natural water system.

4. The method of claim 2, wherein the external water system is a waste water system.

5. The method of claim 1, wherein the water carbon condition comprises a weight measurement of at least CO₂, HCO₃, and CO₃.

6. The method of claim 1, wherein determining the DIC concentration state comprises:
determining a set of derived variables from the sensor data;
determining a set of preliminary dissolved inorganic carbon concentration values from a set of distinct modeling methods based on the derived variables; and
determining the dissolved concentration state through convergent analysis of the set of preliminary dissolved concentration values.

7. The method of claim 6, wherein determining a set of preliminary dissolved inorganic carbon concentration values from a set of distinct modeling methods based on the derived variables comprises:
determining a salinity from the water conductivity,
determining a set of solubility constants of CO₂ in water,
determining an alkalinity from at least the water conductivity,
determining a partial pressure of CO₂ in the atmosphere from pressure and CO₂ concentration in the gas portion
determining CO₂ dissolved in the water from the partial pressure CO₂ and a solubility constant for CO₂ from the set of solubility constants; and
wherein determining the set of preliminary dissolved inorganic carbon concentration values from a set of distinct modeling methods based on the derived variables comprises using a set of distinct modeling methods comprising a modeling method based on alkalinity and pH, a modeling method based on CO₂ and pH, and a modeling method based on alkalinity and CO₂.

8. The method of claim 1, further comprising detecting equilibrium state of the water.

9. The method of claim 1, further comprising:
at a second monitoring system, enclosing a second volume of water within a second receptacle of the second monitoring system, with the water at a filled level that leaves a defined gas portion within the second receptacle, wherein the second monitoring system is downstream from the first monitoring system within a water system;
collecting sensor data from the second volume of water and the defined gas portion within the second receptacle comprising:
measuring carbon concentration of the gas portion of the second monitoring system, and
measuring water conditions of the second monitoring system including at least water temperature, water conductivity, and water pH;
after the water of the second monitoring system reaches an equilibrium state within the second receptacle, determining dissolved inorganic carbon concentration state at the second monitoring system based on the carbon concentration, temperature, water conductivity, and water pH; and
comparing dissolved inorganic carbon concentration state at the first monitoring system to the second monitoring system and determining a change in dissolved inorganic carbon concentration states.

10. The method of claim 10, further comprising adding supplement to the water system and regulating the adding of supplement based on the determined change in dissolved inorganic carbon concentration states.

11. The method of claim 1, further comprising adding supplement that is an alkaline, and regulating the adding of the supplement to satisfy a target water carbon condition.

12. The method of claim 1, wherein the target water carbon conditions comprise a pH less than 8.5.

13. The method of claim 1, further comprising dispensing gaseous carbon dioxide through the water, and regulating the dispensing of the gaseous dioxide to satisfy a target water carbon condition.

14. A system comprising:
a measuring receptacle;
a sensor system;
a control system with or more computer-readable mediums storing instructions that, when executed by the one or more computer processors, cause the control system to perform operations comprising:
enclosing water within the measuring receptacle of the first monitoring system, with the water at a filled level that leaves a defined gas portion within the measuring receptacle;
collecting sensor data from the water and the defined gas portion comprising:
measuring carbon concentration of the gas portion, and
measuring water conditions including at least water temperature, water conductivity, and water pH;
after the water reaches an equilibrium state within the receptacle, determining dissolved inorganic carbon concentration state based on the carbon concentration, temperature, water conductivity, and water pH.

15. The system of claim 14, wherein the measuring receptacles comprises at least one opening controlled to enclose the water.

16. The system of claim 14, wherein the sensor system comprises a pressure sensor 121 and a spectroscopic sensor integrated within the gas portion of the measuring receptacle, and a temperature sensor, a conductivity sensor and a pH sensor integrated within the water.

17. The system of claim 14, further comprising a carbon modification system; and wherein the instructions further cause the control system to perform operations comprising: at the carbon modification system, adding supplement to the water system and regulating the adding of the supplement based on the determined change in dissolved inorganic carbon concentration states.

18. A non-transitory computer-readable medium storing instructions that, when executed by one or more computer processors of a computing platform, cause the computing platform to perform operations comprising:
at a first monitoring system, enclosing water within a receptacle of the first monitoring system, with the water at a filled level that leaves a defined gas portion within the receptacle;
collecting sensor data from the water and the defined gas portion comprising:
measuring carbon concentration of the gas portion, and
measuring water conditions including at least water temperature, water conductivity, and water pH;
after the water reaches an equilibrium state within the receptacle, determining dissolved inorganic carbon concentration state based on the carbon concentration, temperature, water conductivity, and water pH.

19. The non-transitory computer-readable medium of claim 18, further comprising adding supplement to the water system and regulating the adding of supplement based on the determined change in dissolved inorganic carbon concentration states.
